(19) United States
(12) Patent Application Publication
(10) Pub. No.: US 2022/0404369 A1
(43) Pub. Date: Dec. 22, 2022

(54) SYSTEMS AND METHODS FOR QUANTIFYING AND MODIFYING PROTEIN VISCOSITY

(71) Applicant: Regeneron Pharmaceuticals, Inc., Tarrytown, NY (US)

(72) Inventors: Xiaobin Xu, Tarrytown, NY (US); Aming Zhang, Tarrytown, NY (US)

(21) Appl. No.: 17/684,674

(22) Filed: Mar. 2, 2022

Related U.S. Application Data
(60) Provisional application No. 63/156,217, filed on Mar. 3, 2021.

(51) Int. Cl. G01N 33/68; C07K 1/36; G01N 1/40

(52) U.S. Cl. CPC G01N 33/6848; G01N 33/6854; C07K 1/36; G01N 1/4005; G01N 2001/4016; G01N 2458/15; G01N 2560/00

(57) ABSTRACT

Systems and methods for determining regions of proteins that contribute to self-association of the protein are provided. Methods for modifying the self-association of concentrated protein formulations are also provided.

This application claims priority to U.S. application Ser. No. 63/156,217, filed Mar. 3, 2021, which is hereby incorporated by reference.

### TECHNICAL FIELD OF THE INVENTION

The invention is generally related to methods for predicting viscosity of high concentration therapeutic antibodies.

### BACKGROUND OF THE INVENTION

Monoclonal antibodies are a rapidly growing class of biological therapeutics. Monoclonal antibodies have a wide range of indications including inflammatory diseases, cancer, and infectious diseases. The number of commercially available monoclonal antibodies is increasing at a rapid rate, with ˜70 monoclonal antibody products predicted to be on the market by 2020 (Ecker, D.M, et al., mAbs, 7:9-14 (2015)).

Currently, the most commonly utilized route of administration of therapeutic antibodies is intravenous (IV) infusion. However, subcutaneous injection is being increasingly used for patients with chronic diseases who require frequent dosing. Ready-to-use pre-filled syringes or auto-injector pens allow patients to self-administer therapeutic antibodies. Antibody formulations for subcutaneous injection are typically more concentrated than IV infusion since subcutaneous injection is one bolus administration (typically 1-1.5 mL) in contrast to a slow infusion of antibody over time in the case of IV infusion.

A common challenge encountered with the production of highly concentrated therapeutic monoclonal antibodies is high viscosity (Tomar, D.S., et al., mAbs, 8:216-228 (2016)). High viscosity can cause increased injection time and increased pain at the site of the injection. In addition to problems with administration, highly viscous antibodies also pose problems during bioprocessing of the antibody solution. High viscosity can increase processing time, destabilize the drug product, and increase manufacturing costs. Short range electrostatic and/or hydrophobic protein-protein interactions and electroviscous effects can influence concentration-dependent viscosity behavior of antibodies.

Characterizing the conformation and structural dynamics of an antibody can be a major analytical challenge. Many available structural techniques are either highly sophisticated, requiring very specialized skills and large amounts of sample (>μM quantities), or are of low resolution, making detailed structural analysis difficult. As a result, it is desirable to have techniques available that can probe protein structure with low sample requirements, good resolution, and relatively fast turnaround time.

Therefore, it is an object of the invention to provide methods for identifying regions of proteins that contribute to the viscosity of formulations of that protein.

It is another object of the invention to provide methods for modifying viscosity of concentrated protein solutions.

### SUMMARY OF THE INVENTION

Systems and methods for determining regions of proteins that contribute to the viscosity of formulations of those proteins are provided. Methods for modifying the viscosity of concentrated protein formulations are also provided.

Embodiments provide methods for identifying regions in a protein that contribute to the viscosity of the protein by microdialysing samples of the protein in a microdialysis cartridge against a buffer containing deuterium for at least two different time periods. The microdialysis is subsequently quenched. The quenched samples are then analyzed using an hydrogen/deuterium exchange mass spectrometry system to determine regions of the protein in the sample that have reduced levels of deuterium relative to other regions of the protein. The regions of the protein that have reduced levels of deuterium contribute to the viscosity of the protein.

In certain embodiments, the samples of protein have a concentration of between 10 mg/mL to 200 mg/mL of the protein.

In some embodiments, the samples of protein are microdialysed in a buffer having a pH between 5.0 and 7.5. A preferred buffer for the samples of protein is 10 mM Histidine at pH 6.0. An exemplary deuterium containing buffer includes deuterium in 10 mM Histidine at pH 6.0. Typically, the microdialysis is performed at 2 to 6° C., preferably at 4° C. In some embodiments the microdialysis is performed at 20 to 25° C. Different samples can be dialysed for different lengths of time, for example one sample can be dialysed for 4 hours and another sample can be microdialysed for 24 hours. In some embodiments, the samples are dialysed for 30 min., 4 hours, 24 hours or overnight, i.e., 26 hours.

In certain embodiments, the quenching step is typically performed at −2 to 2° C. for 1 to 5 minutes.

In some embodiments, the method includes the step of digesting the protein into peptides before mass spectrometry analysis.

Other embodiments provide methods of modifying the viscosity of a protein drug, by identifying regions of the protein drug that contribute to the viscosity of the protein drug according to the disclosed methods and modifying the regions of the protein drug that are identified as contributing to the viscosity of the protein drug to modify the viscosity of the protein drug. The regions identified as contributing to the viscosity of the drug can be modified by substituting one or more amino acids in the at least one region to reduce or increase the viscosity as desired.

Other embodiments provide methods for identifying regions in proteins that contribute to self-association of proteins, comprising: microdialysing samples of protein of interest in a microdialysis cartridge against a buffer comprising deuterium for at least two different time periods; subsequently quenching the microdialysis of the samples; and analyzing the quenched samples in an hydrogen/deuterium exchange mass spectrometry system to determine surface charge distributions and hydrophobicity in regions of the protein in the sample that exhibit reduced levels of deuterium relative to other regions of the protein, wherein regions of the protein that exhibit reduced levels of deuterium contribute to self-association of the proteins. The proteins can be monoclonal antibodies, including but not limited to the antibodies described herein. The proteins also can be Fc-fusion proteins, including but not limited to the Fc-fusion proteins described herein.

The protein or protein drug can be an antibody, a fusion protein, a recombinant protein, or a combination thereof. In some embodiments, the protein drug is a concentrated monoclonal antibody.

Conditions, concentrations, timing and steps can be selected by the person skilled in the art based upon the description contained herein.

### BRIEF DESCRIPTION OF THE DRAWINGS

FIG. 1A is a line graph showing viscosity (cP) of mAb1 as a function of concentration (mg/mL). FIG. 1B is a line graph showing viscosity (cP) of mAb2 as a function of concentration (mg/mL).

FIG. 2A-2F is a schematic of an exemplary microdialysis based HDX-MS protocol. Microdialysis cartridges (FIG. 2A) are obtained, D₂O buffer is added to a deep-well plate (FIG. 2B), samples are loaded into the microdialysis cartridges (FIG. 2C), the microdialysis cartridges are loaded into the deep-well plate (FIG. 2D), samples are incubated in the D₂O buffer for various time points (FIG. 2E), and the samples are removed for MS analysis (FIG. 2F).

FIGS. 3A-3F are exemplary spectrograms of deuterium uptake over time in non-CDR mAb1 samples at 15 mg/mL concentrations (FIGS. 3A-3C) and 120 mg/mL concentrations (FIGS. 3D-3F) 0 hours (FIGS. 3A and 3D), 4 hours (FIGS. 3B and 3E), or 24 hours (FIGS. 3C and 3F) after deuterium incubation. FIGS. 3G-3L are spectrograms of deuterium uptake over time in non-CDR mAb1 samples at 15 mg/mL concentrations (FIGS. 3G-3I) and 120 mg/mL concentrations (FIGS. 3J-3L) 0 hours (FIGS. 3G and 3J), 4 hours (FIGS. 3H and 3K), or 24 hours (FIGS. 3I and 3L) after deuterium incubation. FIGS. 3M and 3N are deuterium uptake plots showing deuterium uptake % versus time (hrs) for 15 mg/mL (♦) and 120 mg/mL (▪) for mAb1 HC36-47 and mAb1 LC48-53.

FIGS. 4A-4B and 4E-4F are butterfly plots showing relative deuterium uptake in heavy chain CDR regions for mAb1 (FIGS. 4A and 4E) and mAb2 (FIGS. 4B and 4F) after 4 hours or 24 hours of deuterium incubation. The top plots represent 120 mg/mL sample concentration and the bottom plots represent 15 mg/mL sample concentration. The X axis represents peptide number and the Y axis represents differential deuterium uptake (%). FIG. 4C-4D and 4G-4H are residual plots showing relative deuterium uptake in heavy chain CDR regions for mAb 1 (FIGS. 4C and 4G) and mAb2 (FIGS. 4D and 4H) after 4 hours or 24 hours of deuterium incubation. The top plots represent 120 mg/mL sample concentration and the bottom plots represent 15 mg/mL sample concentration. The X axis represents peptide number and the Y axis represents differential deuterium uptake (%). FIGS. 4G-4H are residual plots of deuterium uptake in mAb1 light chain (FIG. 4G) and mAb2 light chain (FIG. 4H) after 4 hours or 24 hours of incubation. The X axis represents peptide number and the Y axis represents differential deuterium uptake (%).

FIG. 5A is a line graph of deuterium uptake (%) versus time (hours) for mAb1 HC CDR1 peptide 30-33. FIG. 5B is a line graph of deuterium uptake (%) versus time (hours) for mAb2 HC CDR1 peptide 31-34. FIG. 5C is a line graph of deuterium uptake (%) versus time (hours) for mAb1 HC CDR2 peptide 50-54. FIG. 5D is a line graph of deuterium uptake (%) versus time (hours) for mAb2 HC CDR2 peptide 50-53. FIG. 5E is a line graph of deuterium uptake (%) versus time (hours) for mAb1 HC CDR2 peptide 101-104. FIG. 5F is a line graph of deuterium uptake (%) versus time (hours) for mAb2 HC CDR3 peptide 99-103. FIG. 5G is a line graph of deuterium uptake (%) versus time (hours) for mAb1 LC CDR2 peptide 48-53. FIG. 5H is a line graph of deuterium uptake (%) versus time (hours) for mAb2 LC CDR2 peptide 47-52. FIG. 51 is a line graph of deuterium uptake (%) versus time (hours) for mAb1 HC CDR2 HC non-CDR peptide 36-47. FIG. 5J is a line graph of deuterium uptake (%) versus time (hours) for mAb2 HC non-CDR peptide 36-47.

FIG. 6A is shows deuterium uptake measured by HDX-MS plotted onto a homology model of mAb 1. FIG. 6B is a zoom-in view of the Fab domain of mAb 1. CDR regions are shown in balls. Regions with differential deuterium uptakes ≥10% (absolute value) are indicated by arrows without significantly differential deuterium uptake (<10%, absolute value). FIG. 6C is a zoom-in view of the Fab domain surface patches of mAb 1 and FIG. 6D is a zoom-in view of the Fab domain surface patches of mAb2. CDR regions are shown as balls. Hydrophobic patches are indicated with an arrow. Positive patches indicated with an arrow. Negative patches are indicated with an arrow.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “protein” refers to a molecule comprising two or more amino acid residues joined to each other by a peptide bond. Protein includes polypeptides and peptides and may also include modifications such as glycosylation, lipid attachment, sulfation, gamma-carboxylation of glutamic acid residues, alkylation, hydroxylation and ADP-ribosylation. Proteins can be of scientific or commercial interest, including protein-based drugs, and proteins include, among other things, enzymes, ligands, receptors, antibodies and chimeric or fusion proteins. Proteins are produced by various types of recombinant cells using well-known cell culture methods, and are generally introduced into the cell by transfection of genetically engineering nucleotide vectors (e.g., such as a sequence encoding a chimeric protein, or a codon-optimized sequence, an intronless sequence, etc.), where the vectors may reside as an episome or be intergrated into the genome of the cell.

“Antibody” refers to an immunoglobulin molecule consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has a light chain variable region and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. The term antibody also includes bispecific antibody, which includes a heterotetrameric immunoglobulin that can bind to more than one different epitope. Bispecific antibodies are generally described in US Patent Application Publication No. 2010/0331527.

A “CDR” or complementarity determining region is a region of hypervariability interspersed within regions that are more conserved, termed “framework regions” (FR). The FRs may be identical to the human germline sequences, or may be naturally or artificially modified.

As used herein, “viscosity” refers to the rate of transfer of momentum of liquid. It is a quantity expressing the magnitude of internal friction, as measured by the force per unit area resisting a flow in which parallel layers unit distance apart has unit speed relative to one another. In liquids, viscosity refers to the “thickness” of a liquid.

The term “HDX-MS” refers to hydrogen/deuterium exchange mass spectrometry.

As used herein, “dialysis” is a separation technique that facilitates the removal of small, unwanted compounds from macromolecules in solution by selective and passive diffusion through a semi-permeable membrane. A sample and a buffer solution (called the dialysate, usually 200 to 500 times the volume of the sample) are placed on opposite sides of the membrane. Sample molecules that are larger than the membrane-pores are retained on the sample side of the membrane, but small molecules and buffer salts pass freely through the membrane, reducing the concentration of those molecules in the sample. Once the liquid-to-liquid interface (sample on one side of the membrane and dialysate on the other) is initiated, all molecules will try to diffuse in either direction across the membrane to reach equilibrium. Dialysis (diffusion) will stop when equilibrium is achieved. Dialysis systems are also used for buffer exchange.

The term “microdialysis” refers to the dialysis of samples having a volume of less than one milliliter.

“D₂O ” is an abbreviation for deuterated water. It is also known as heavy water or deuterium oxide. D₂O contains high amounts of the hydrogen isotope deuterium instead of the common hydrogen isotope that makes up most of the hydrogen in normal water. Deuterium is an isotope of hydrogen that is twice as heavy due to an added neutron.

### II. Methods for Identifying Regions of Proteins that Contribute to Viscosity

The development of highly concentrated therapeutic monoclonal antibodies is paramount for subcutaneous delivery of monoclonal antibody therapeutics. However, high viscosity is a concern in the production of concentrated monoclonal antibody therapeutics. There is a need to develop computational and experimental tools to rapidly and efficiently determine the concentration-dependent viscosity behavior of candidate therapeutics early in the development process.

### A. Microdialysis-Hydrogen/Deuterium Exchange Mass Spectrometry

During the course of development, a therapeutic monoclonal antibody can exhibit unusually high viscosity, for example at concentrations >100 mg/mL when compared to other similar monoclonal antibodies. This may be due to the characteristic short range electrostatic and/or hydrophobic protein-protein interactions of the monoclonal antibody under high concentrations. Hydrogen/deuterium exchange mass spectrometry (HDX-MS) is a useful tool to investigate protein conformation, dynamics, and interactions. However, the conventional dilution labeling HDX-MS analysis has a limitation on analyzing unusual behaviors that only occur at high protein concentrations. In order to probe protein-protein interactions governing high viscosity of monoclonal antibodies at a high protein concentration with HDX-MS, a passive, microdialysis based HDX-MS method to achieve HDX labeling without D₂O buffer dilution was developed, which allows for the profiling of characteristic molecular interactions at different protein concentrations. The use of a microdialysis plate significantly reduced the consumption of samples and D₂O compared to the traditional dialysis devices. This method was applied to investigate protein-protein interactions at a high concentration of monoclonal antibodies which have very high viscosity.

Proteins with high viscosity behavior can be optimized to reduce or eliminate the high viscosity behavior. Methods of optimizing protein drugs or antibodies include but are not limited to optimizing the amino acid sequence to reduce viscosity, altering the pH or salt content of the formulation, or adding an excipient.

In one embodiment, multiple therapeutic protein or antibody formulations can be tested to determine the most promising candidate to move forward in production. High and low concentration samples of each protein or antibody are produced. In one embodiment, a high protein or antibody concentration is >50 mg/mL. The high concentration can be 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, or >200 mg/mL. In one embodiment, a low antibody concentration is <15 mg/mL. The low concentration can be 15 mg/mL, 10 mg/mL, 9 mg/mL, 8 mg/mL, 7 mg/mL, 6 mg/mL, 5 mg/mL, 4mg/mL, 3 mg/mL, 2 mg/mL, 1 mg/mL, 0.5 mg/mL, or <0.5 mg/mL.

More details in the steps of the disclosed methods are provided below.

### 1. Hydrogen/Deuterium Exchange

Hydrogen/deuterium exchange is a phenomenon in which hydrogen atoms at labile positions in proteins spontaneously change places with hydrogen atoms in the surrounding solvent which contains deuterium ions (Houde, D. and Engel, J. R., Methods Mol Biol, 988:269-289 (2013)). HDX takes advantage of the three types of hydrogens in proteins: those in carbon-hydrogen bonds, those in side-chain groups, and those in amide functional groups (also called backbone hydrogens). The exchange rates of hydrogens in carbon-hydrogen bonds are too slow to observe, and those of side-chain hydrogens (e.g., OH, COOH) are so fast that they back-exchange rapidly when the reaction is quenched in H₂O -based solution, and the exchange is not registered. Only the backbone hydrogens are useful for reporting protein structure and dynamics because their exchange rates are measurable and reflect hydrogen bonding and solvent accessibility. Amide hydrogens play a key role in the formation of secondary and tertiary structure elements. Measurements of their exchange rates can be interpreted in terms of the conformational dynamics of individual higher-order structural elements as well as overall protein dynamics and stability.

Exchange rates reflect on the conformational mobility, hydrogen bonding strength, and solvent accessibility in protein structure. Information about protein conformation and, most importantly, differences in protein conformation between two or more forms of the same protein can be extracted by monitoring the exchange reaction. The exchange rate is temperature dependent, decreasing by approximately a factor of ten as the temperature is reduced from 25° C. to 0° C. Consequently, under pH 2-3 and at 0° C. (commonly referred to as “quench conditions”) the half-life for amide hydrogen isotopic exchange in an unstructured polypeptide is 30-90 min, depending on the solvent shielding effect caused by the side chains. Hydrogen has a mass of 1.008 Da and deuterium (the second isotope of hydrogen) has a mass of 2.014 Da, hydrogen exchange can be followed by measuring the mass of a protein with a mass spectrometer.

In one embodiment, hydrogen/deuterium exchange rate is used to determine viscosity behavior of protein or antibody therapeutics.

### 2. Microdialysis

Classical continuous HDX labeling via dilution is not applicable in the analysis of highly concentrated protein solutions. One embodiment herein provides an alternative method of HDX labeling for the use with high concentration protein solutions. HDX labeling in a microdialysis plate facilitates the analysis of highly concentrated protein solutions. In addition, the use of a microdialysis plate reduces the consumption of samples and D₂O compared to traditional dialysis devices (Houde, D., et al., J Am Soc Mass Spectrom, 27(4):669-76 (2016)). The microdialysis plate can be a commercially available microdialysis plate, for example Pierce™ 96-well Microdialysis Plate.

In one embodiment, microdialysis HDX exchange is used to analyze highly concentrated protein solutions. The samples are loaded into the microdialysis cartridge of the microdialysis plate. D₂O buffer is added to a deep-well plate or other suitable vessel. The microdialysis cartridges containing the protein samples are added to the buffer and allowed to incubate for at least 4 hours. The samples can incubate for 0.5, 1, 2, 3, 4, 5, 6, 7, 8, 9, 10, 11, 12, 13, 14, 15, 16, 17, 18, 19, 20, 21, 22, 23, 24, or more than 24 hours. The dialysis system allows for passive diffusion of the buffer into the cartridge containing the sample so as to not dilute out the sample as is common in traditional continuous HDX labeling wherein large quantities of buffer are required. During the incubation step, deuterium in the D₂O buffer enters into the cartridge containing the sample and is exchanged with hydrogens in the backbone amides of the protein samples. After the incubation step, samples are collected from the microdialysis cartridge.

### 3. Sample Preparation

Once the dialyzed samples are removed from the microdialysis cartridge, the HDX reaction can be terminated by quenching the samples. In one embodiment, quenching is achieved by adding quench buffer to the samples. The quenching buffer can contain 6M GlnHCl and 0.6M TCEP in H2O, pH 2.5. In one embodiment, the quenching buffer contains 8 M Urea, 0.6M TCEP in H₂0, pH 2.5. In another embodiment, the pH of the final quenched solution is 2.5.

In one embodiment, decreasing the reaction temperature can also quench the HDX reaction. The reaction can be carried out at 0° C. The exchange rate decreases by a factor of ten as the temperature is reduced from 25° C. to 0° C. In one embodiment, the quenching reaction is carried out at or below 0° C.

After quenching, the samples can be diluted for downstream mass spec analysis. Samples can be diluted in 0.1% formic acid (FA) in H₂O or any other suitable diluent for use in mass spectrometry. The samples are then processed by a mass spectrometer.

### 4. Mass Spectrometry

Mass spectrometry is used for determining the mass shifts induced by the exchange of hydrogen by deuterium (or vice versa) over time. Hydrogen has a mass of 1.008 Da and deuterium has a mass of 2.014 Da, therefore hydrogen exchange can be followed by measuring the mass of a protein with a mass spectrometer. Proteins or antibodies that have incorporated deuterium will have an increased mass compared to the native protein or antibody that has not been incubated in D₂O. Generally, the level of exchanged hydrogen reflects the flexibility, solvent accessibility, and hydrogen bonding interactions in protein structures.

In some embodiments on-line digestion is employed to cleave larger proteins or antibodies into smaller fragments or peptides. Commonly used enzymes for on-line digestion include but are not limited to pepsin, trypsin, trypsin/Lys-C, rLys-C, Lys-C, and Asp-N.

In one embodiment, the digested proteins or antibodies are subjected to mass spectrometry analysis. Methods of performing mass spectrometry are known in the art. See for example (Aeberssold, M., and Mann, M., Nature, 422:198-207 (2003)) Commonly utilized types of mass spectrometry include but are not limited to tandem mass spectrometry (MS/MS), electrospray ionization mass spectrometry, liquid chromatography-mass spectrometry (LC-MS), and Matrix-assisted laser desorption /ionization (MALDI).

### III. Methods for Modifying Protein Viscosity

One embodiment provides a method of modifying the viscosity of a protein drug, by identifying regions of the protein drug that contribute to the viscosity of the protein drug according to the disclosed methods and modifying the regions of the protein drug that are identified as contributing to the viscosity of the protein drug to modify the viscosity of the protein drug. The regions identified as contributing to the viscosity of the drug can be modified by substituting one or more amino acids in the at least one region to reduce or increase the viscosity as desired.

For example, the light chain, heavy chain, or complementarity determining regions of an antibody can be modified to reduce the viscosity of concentrated formulations of the antibody. An exemplary concentrated formulation has a concentration of antibody that is greater than 50 mg/mL, preferably 100 mg/mL or greater.

Other modifications of the protein or antibody drug include chemical modifications to amino acids in the region of the protein or antibody determined to contribute to the viscosity of the protein or antibody drug.

In one embodiment the protein, antibody, or drug product is or contains one or more proteins of interest suitable for expression in prokaryotic or eukaryotic cells. For example, the protein of interest includes, but is not limited to, an antibody or antigen-binding fragment thereof, a chimeric antibody or antigen-binding fragment thereof, an ScFv or fragment thereof, an Fc-fusion protein or fragment thereof, a growth factor or a fragment thereof, a cytokine or a fragment thereof, or an extracellular domain of a cell surface receptor or a fragment thereof. Proteins of interest may be simple polypeptides consisting of a single subunit, or complex multisubunit proteins comprising two or more subunits. The protein of interest may be a biopharmaceutical product, food additive or preservative, or any protein product subject to purification and quality standards.

In some embodiments, the protein of interest is an antibody, a human antibody, a humanized antibody, a chimeric antibody, a monoclonal antibody, a multispecific antibody, a bispecific antibody, an antigen binding antibody fragment, a single chain antibody, a diabody, triabody or tetrabody, a dual-specific, tetravalent immunoglobulin G-like molecule, termed dual variable domain immunoglobulin (DVD-IG), an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody. In another embodiment, the antibody comprises a chimeric hinge. In still other embodiments, the antibody comprises a chimeric Fc. In one embodiment, the antibody is a chimeric IgG2/IgG4 antibody. In one embodiment, the antibody is a chimeric IgG2/IgG1 antibody. In one embodiment, the antibody is a chimeric IgG2/IgG1/IgG4 antibody.

In some embodiments, the antibody is selected from the group consisting of an anti-Programmed Cell Death 1 antibody (e.g. an anti-PD1 antibody as described in U.S. Pat. Appln. Pub. No. US2015/0203579A1), an anti-Programmed Cell Death Ligand-1 (e.g., an anti-PD-L1 antibody as described in in U.S. Pat. Appln. Pub. No. US2015/0203580A1), an anti-Dll4 antibody, an anti-Angiopoetin-2 antibody (e.g., an anti-ANG2 antibody as described in U.S. Pat. No. 9,402,898), an anti-Angiopoetin-Like 3 antibody (e.g., an anti-AngPtl3 antibody as described in U.S. Pat. No. 9,018,356), an anti-platelet derived growth factor receptor antibody (e.g., an anti-PDGFR antibody as described in U.S. Pat. No. 9,265,827), an anti-Erb3 antibody, an anti-Prolactin Receptor antibody (e.g., anti-PRLR antibody as described in U.S. Pat. No. 9,302,015), an anti-Complement 5 antibody (e.g., an anti-C5 antibody as described in U.S. Pat. Appln. Pub. No US2015/0313194A1), an anti-TNF antibody, an anti-epidermal growth factor receptor antibody (e.g., an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (e.g., an anti-PCSK9 antibody as described in U.S. Pat. No. 8,062,640 or U.S. Pat. No. 9,540,449), an Anti-Growth and Differentiation Factor-8 antibody (e.g. an anti-GDF8 antibody, also known as anti-myostatin antibody, as described in U.S. Pat Nos. 8,871,209 or 9,260,515), an anti-Glucagon Receptor (e.g. anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. Appln. Pub. No. US2014/0271681A1 or U.S. Pat Nos. 8,735,095 or 8,945,559), an anti-interleukin 6 receptor antibody (e.g., an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g., anti-IL33 antibody as described in U.S. Pat. Nos. 9,453,072 or 9,637,535), an anti-Respiratory syncytial virus antibody (e.g., anti-RSV antibody as described in U.S. Pat. Appln. Pub. No. 9,447,173), an anti-Cluster of differentiation 3 (e.g., an anti-CD3 antibody, as described in U.S. Pat. Nos. 9,447,173 and 9,447,173, and in U.S. Application No. 62/222,605), an anti-Cluster of differentiation 20 (e.g., an anti-CD20 antibody as described in U.S. Pat. Nos. 9,657,102 and US20150266966A1, and in U.S. Pat. No. 7,879,984), an anti-CD19 antibody, an anti-CD28 antibody, an anti-Cluster of Differentiation-48 (e.g. anti-CD48 antibody as described in U.S. Pat. No. 9,228,014), an anti-Fel d1 antibody (e.g. as described in U.S. Pat. No. 9,079,948), an anti-Middle East Respiratory Syndrome virus (e.g. an anti-MERS antibody as described in U.S. Pat. Appln. Pub. No. US2015/0337029A1), an anti-Ebola virus antibody (e.g. as described in U.S. Pat. Appln. Pub. No. U52016/0215040), an anti-Zika virus antibody, an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. Appln. Pub. No. US2016/0017029 and U.S. Pat. Nos. 8,309,088 and 9,353,176) and an anti-Protein Y antibody. In some embodiments, the bispecific antibody is selected from the group consisting of an anti-CD3 x anti-CD20 bispecific antibody (as described in U.S. Pat. Appln. Pub. Nos. US2014/0088295A1 and US20150266966A1), an anti-CD3 x anti-Mucin 16 bispecific antibody (e.g., an anti-CD3 x anti-Muc16 bispecific antibody), and an anti-CD3 x anti-Prostate-specific membrane antigen bispecific antibody (e.g., an anti-CD3 x anti-PSMA bispecific antibody). In some embodiments, the protein of interest is selected from the group consisting of abciximab, adalimumab, adalimumab-atto, ado-trastuzumab, alemtuzumab, alirocumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cemiplimab, cetuximab, denosumab, dinutuximab, dupilumab, durvalumab, eculizumab, elotuzumab, emicizumab-kxwh, emtansinealirocumab, evinacumab, evolocumab, fasinumab, golimumab, guselkumab, ibritumomab tiuxetan, idarucizumab, infliximab, infliximab-abda, infliximab-dyyb, ipilimumab, ixekizumab, mepolizumab, necitumumab, nesvacumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, sarilumab, secukinumab, siltuximab, tocilizumab, tocilizumab, trastuzumab, trevogrumab, ustekinumab, and vedolizumab.

In some embodiments, the protein of interest is a recombinant protein that contains an Fc moiety and another domain, (e.g., an Fc-fusion protein). In some embodiments, an Fc-fusion protein is a receptor Fc-fusion protein, which contains one or more extracellular domain(s) of a receptor coupled to an Fc moiety. In some embodiments, the Fc moiety comprises a hinge region followed by a CH2 and CH3 domain of an IgG. In some embodiments, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-1RAcP ligand binding region fused to the Il-1R1 extracellular region fused to Fc of hIgG1; see U.S. Pat. No. 6,927,044), or a VEGF trap (e.g., aflibercept or ziv-aflibercept, which comprises the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). In other embodiments, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

In one embodiment, the protein drug is a concentrated monoclonal antibody.

### EXAMPLES

### Example 1. Microdialysis HDX Mass Spectrometry

### Materials and Methods

### Reagents and Chemicals

mAb1 and mAb2 (human IgG4 mAbs) were manufactured by Regeneron Pharmaceuticals, Inc. (Tarrytown, N.Y.). Deuterium oxide (99.9 atom % D), histidine, histidine hydrochloride monohydrate, and guanidine hydrochloride were purchased from Sigma Aldrich (St. Louis, Mo.). Tris (2-carboxyethyl) phosphine hydrochloride (TCEP-HC1), formic acid (FA, sequencing grade), and 96-well microdialysis plate (10 kDa molecular weight cutoff, MWCO) were purchased from Thermo Fisher Scientific (Waltham, Mass.). High purity water was generated using a Milli-Q system from Millipore Sigma (Bedford, Mass.).

Concentration and Viscosity Measurement of mAb1 and mAb2 Samples

The high concentration mAb1 and mAb2 samples (120 mg/mL) were diluted with 10 mM histidine to a series of lower concentrations: 100 mg/mL, 80 mg/mL, 60 mg/mL, 30 mg/mL, and 15 mg/mL (Table 4). The concentration of each diluted sample was measured by a NanoDrop microvolume spectrophotometer from Thermo Fisher Scientific (Waltham, Mass.) and shown in Table 4. The viscosity of each mAb1 and mAb2 sample was measured by Rheosense m-VROC viscometer (San Ramon, Calif.).

TABLE 4
Concentration measurement of mAb1 and
mAb2 samples created by serial dilution

| Serial | Expected Nominal | mAb1 Measured | mAb2 Measured |
|---|---|---|---|
| Dilution | Concentration | Concentration | Concentration |
| Point | (mg/mL) | (mg/mL) | (mg/mL) |
| 1 | Original sample (~120) | No measurement | No measurement |
| 2 | 100 | 104.2 | 102.4 |
| 3 | 80 | 87.0 | 83.4 |
| 4 | 60 | 55.7 | 56.8 |
| 5 | 30 | 29.5 | 32.8 |
| 6 | 15 | 15.7 | 15.3 |

### Dilution Free Microdialysis

mAb 1 and mAb2 were diluted in 10 mM histidine (pH 6.0) to create high concentration samples (120 mg/mL) and low concentration samples (15 mg/mL). 160 μl of each sample was loaded into a microdialysis cartridge. The cartridge was inserted into a deep-well plate containing D₂O buffer and incubated for 4 or 24 hours at 4° C. After incubation, 5 μl of each dialyzed sample was quenched by adding quench buffer to the sample, according to Table 1. Quench buffer contains 6M GlnHCl/0.6 M TCEP in 100% D₂O. The quenching reaction was carried out at 0° C. for 3 minutes. 10 μl of each quenched sample was diluted with 0.1% FA in D₂O, according to Table 1. 70 μl of each sample was loaded onto an HDX system. Table 1. Sample buffers and dilution volumes.

Immediately after, 10 μl of each quenched sample was quickly mixed with the requisite volume of 0.1% FA in H₂O at 0° C. to adjust the protein concentration of each sample to 0.1 μg/μL. Immediately after, each sample was analyzed using a custom HDX-MS system, which consisted of a liquid-cooling HDX autosampler (NovaBioAssays, Woburn, Mass.) for digestion and loading, a UHPLC system (Jasco, Easton, Mass.) for peptide separation, and a Q Exactive Plus Hybrid Quadrupole—Orbitrap Mass Spectrometer (ThermoFisherScientific, Waltham, Mass.) for the peptide mass measurement. In brief, 7 μg of each sample was injected onto an immobilized pepsin/protease XIII column (NovaBioassays, Woburn, Mass.) for online digestion and HPLC separation. The digested peptides were trapped onto a 1.0×50 mm C8 column (NovaBioAssays, Woburn, Mass.) at −9° C. After the column was desalted for 3 min, the trapped peptides were eluted by a 25-min gradient with a UHPLC system (Jasco, Easton, Mass.) at −9° C. Mobile phase A was 0.5% FA/95% water/4.5% acetonitrile, and mobile phase B was 0.1% FA in acetonitrile. The column was initially equilibrated with 100% mobile phase A. Post sample injection and trapping, the gradient began with a 0.5 min hold at 0% mobile phase B followed by an increase to 8% mobile phase B over 2.5 min and an increase to 28% mobile phase B over the next 14 min for peptide separation. The column was then washed by an increase to 95% mobile phase B over 3 min followed by a decrease to 2% mobile phase B over 0.5 min. The gradient ended with a 4.5 min hold at 2% mobile phase B. The separated peptides were analyzed by mass spectrometry in MS and MS/MS modes. The MS parameters were set as follows: resolving power, 70000 (m/z 200) in MS scan and 35000 in MS/MS scan; spray voltage, 3.8 kV; capillary temperature, 325° C.; AGC target, 3e6 in MS scan and 1e5 in MS/MS scan; maximum injection time, 100 ms for MS scan and 50 ms for MS/MS scan; MS/MS loop count, 6; m/z range, 300-1500; and stepped NCE, 15-26-36. The LC-MS/MS data of undeuterated mAb1 and mAb2 samples were searched against a database including mAb1 and mAb2 and their randomized sequence using a Byonic™ search engine (Protein Metrics, Cupertino, Calif.). The identified peptide list was then imported together with the LC-MS data from all deuterated samples into the HDExaminer™ software (Sierra Analytics, Modesto, Calif.) to calculate the deuterium uptakes of individual peptides in each sample. mAb1 and mAb2 homology modelling and protein surface patch analyses were performed using MOE (Version 2019.0102, Chemical Computing Group, Montreal, QC, Canada).

TABLE 1

| | Volume of | Volume of | Injection |
|---|---|---|---|
| Sample | Quench Buffer | Dilution Buffer | Amount |
| 120 mg/mL | 5 μL → 295 μL | 10 μL → 130 μL | 70 μL (7 μg) |
| | (2 mg/mL) | (0.1 mg/mL) | |
| 15 mg/mL | 5 μL → 70 μL | 20 μL → 120 μL | 70 μL (7 μg) |
| | (1 mg/mL) | (0.1 mg/mL) | |

### Results

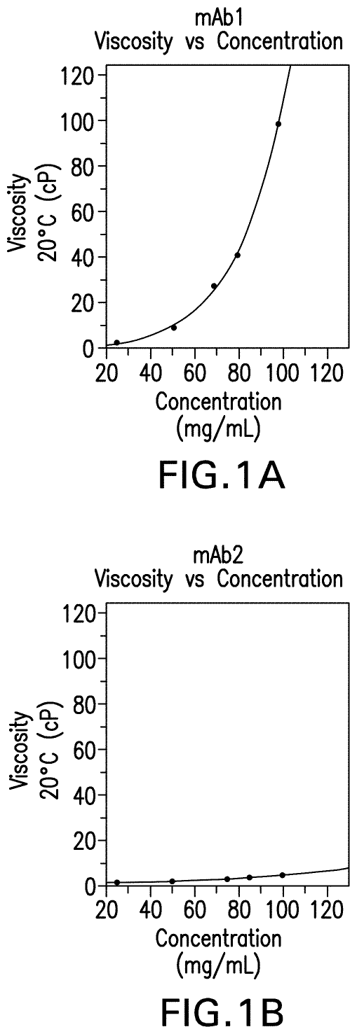

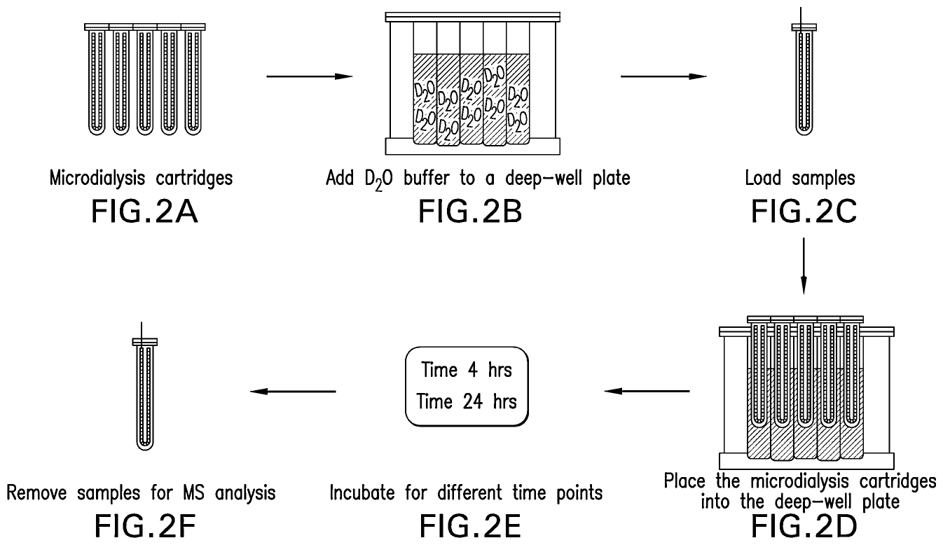

Monoclonal antibody 1 (mAb1) exhibited unusually high viscosity at concentrations >100 mg/mL, when compared to other monoclonal antibodies at the development stage (FIGS. 1A-1B). To probe protein-protein interactions governing the high viscosity of mAb1 at a high protein concentration, a passive, microdialysis based HDX-MS method was developed to achieve HDX labeling without D₂O buffer dilution, which allows profiling molecular interactions at different protein concentrations (FIG. 2A-2F).

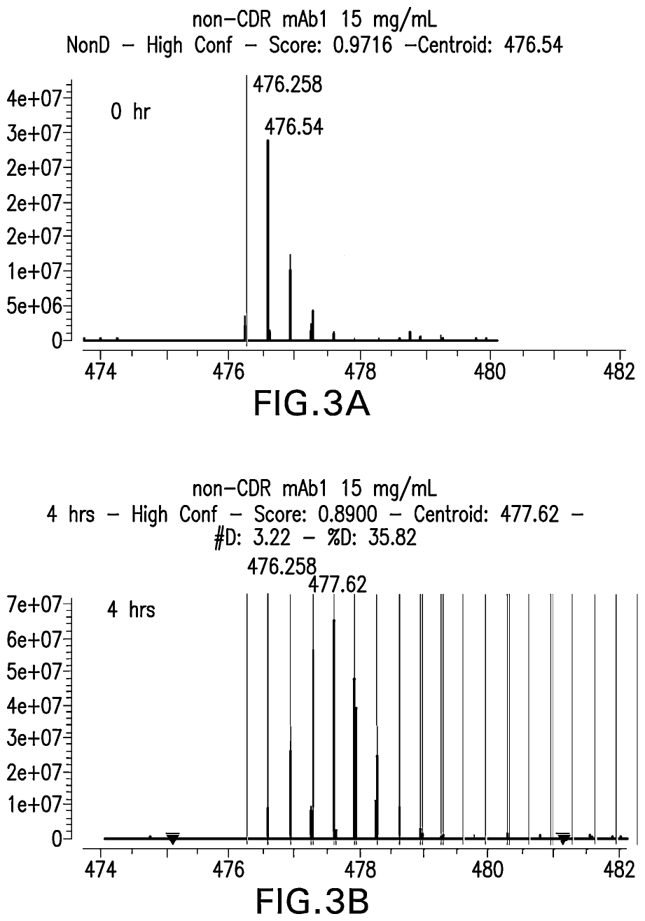

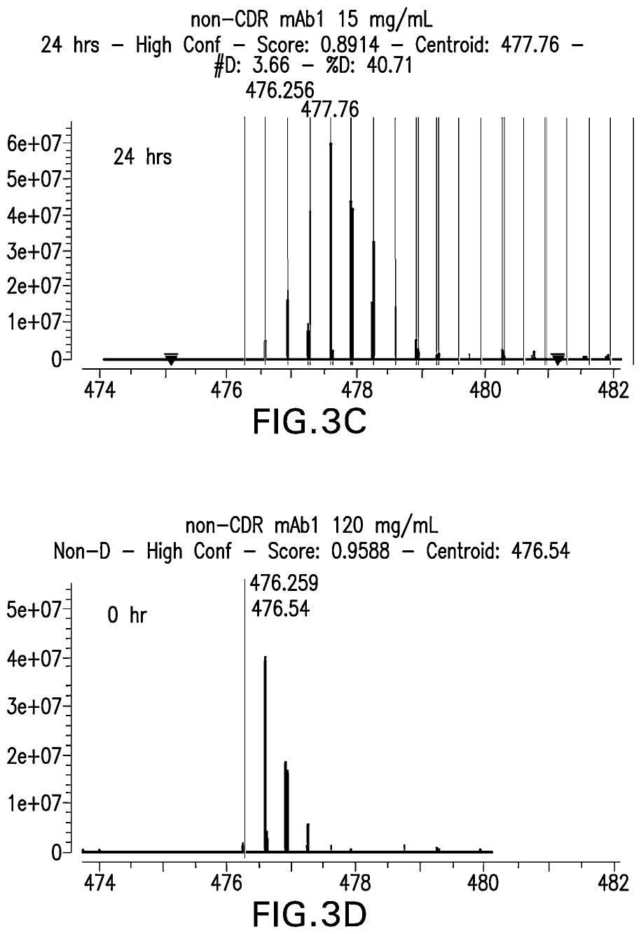

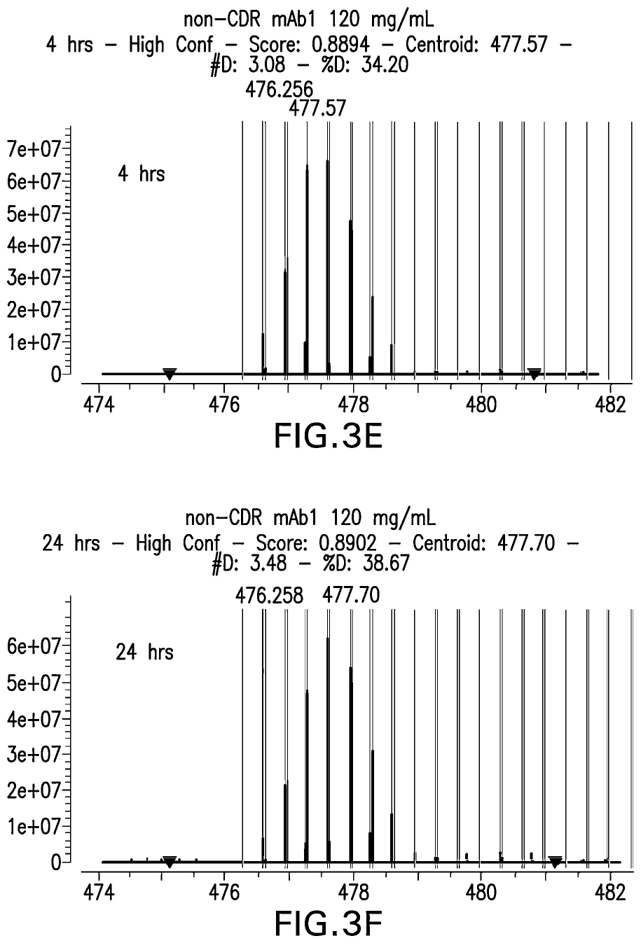

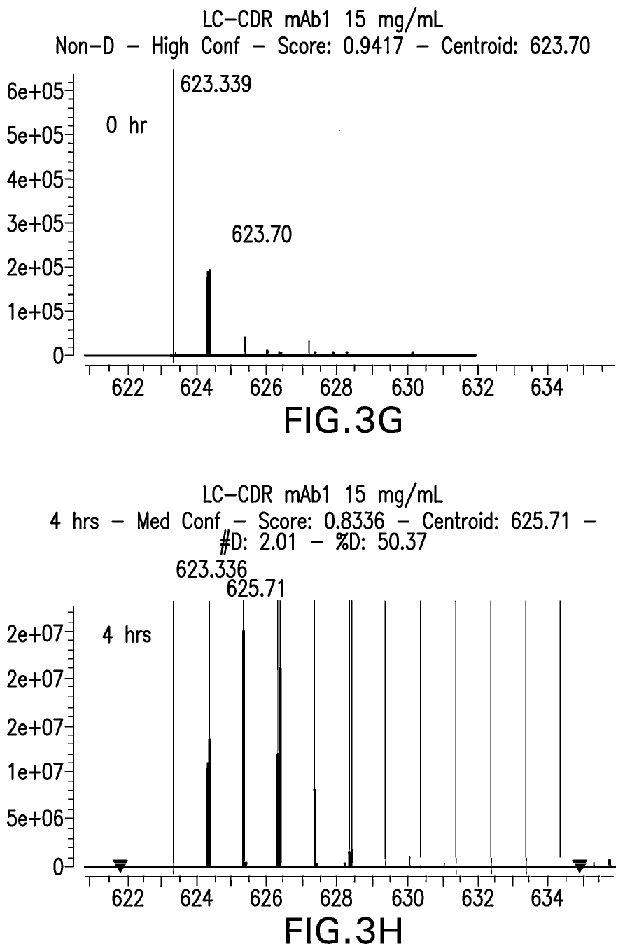

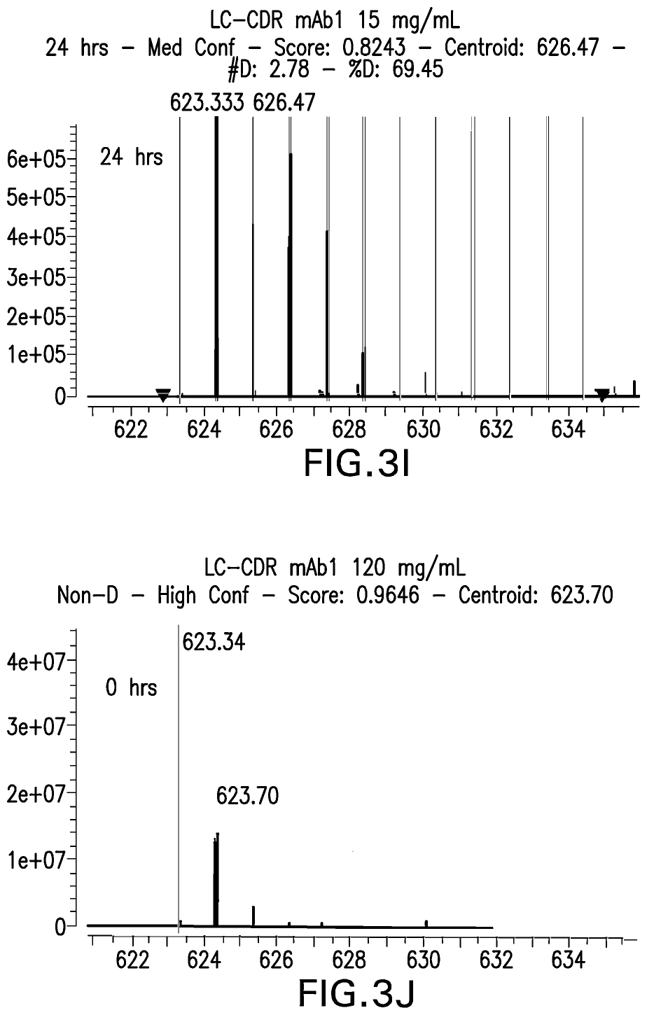

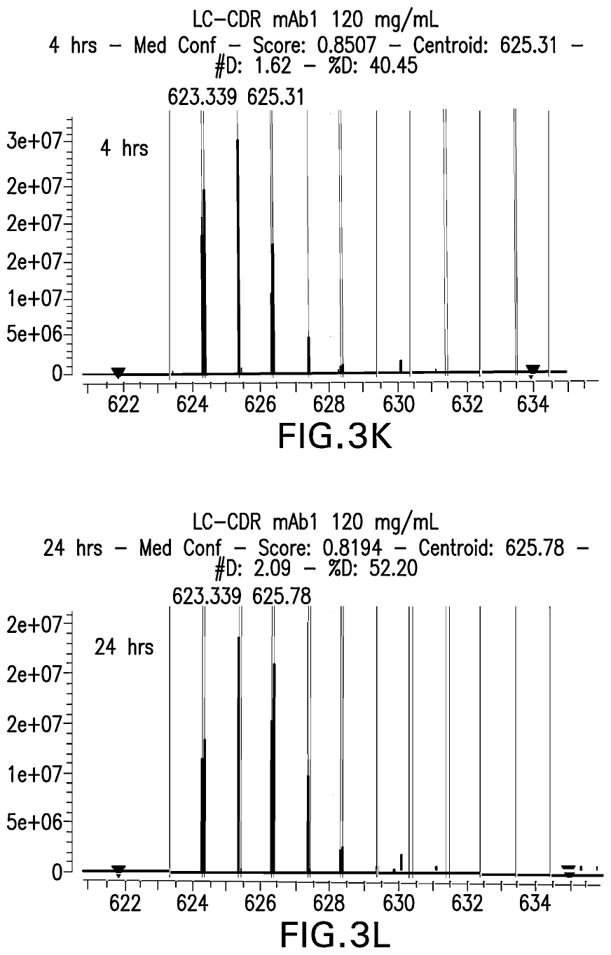

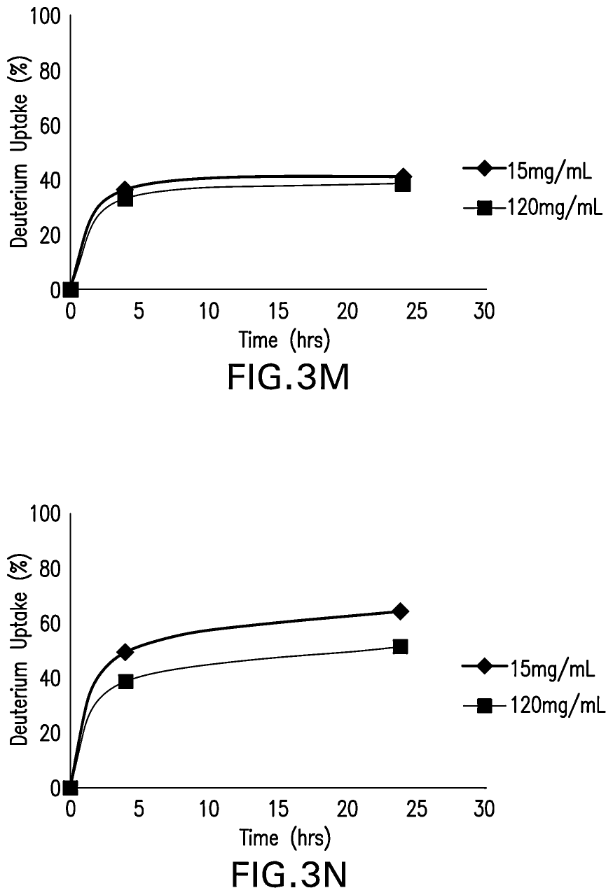

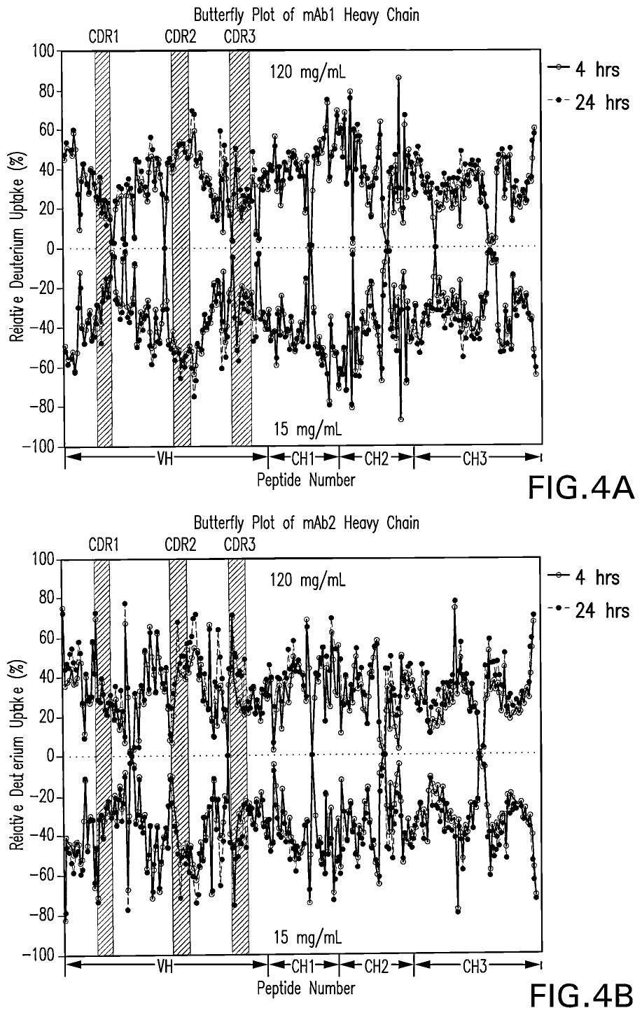

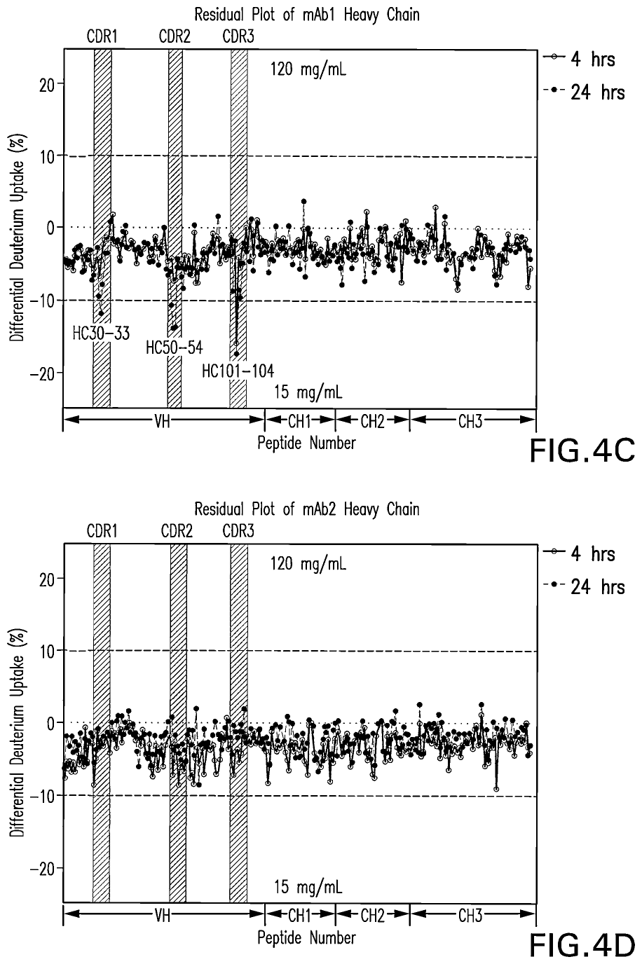

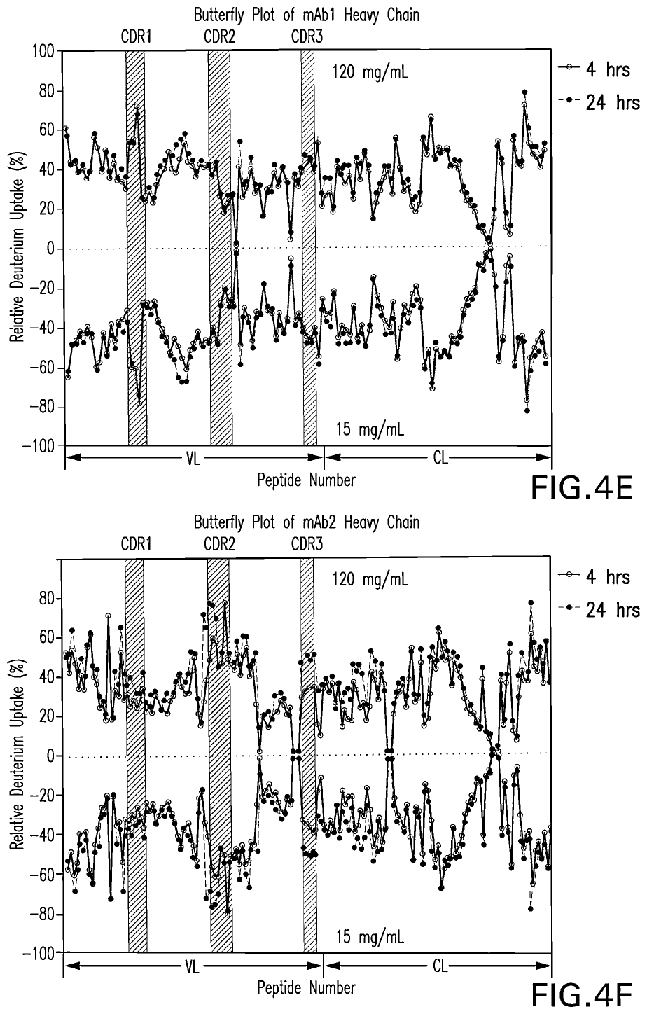

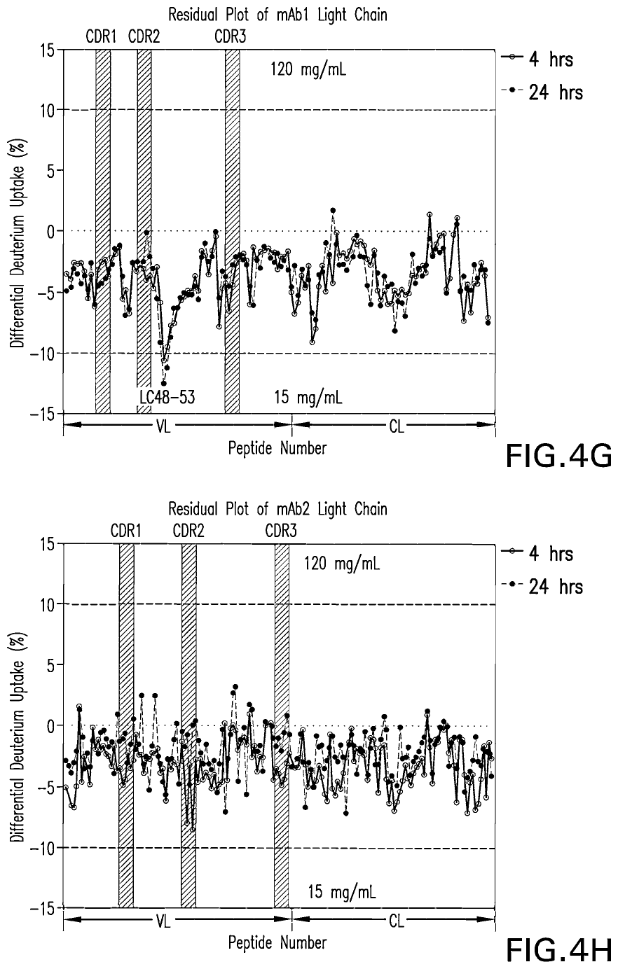

A significant decrease in deuterium was observed in the high concentration samples (120 mg/mL) compared to the control samples (15 mg/mL) at the three heavy chain complementary determining regions and light chain CDR2 for mAb1 (FIGS. 3A-3N, Table 2 and Table 3). This result indicates that these CDRs may be involved in specific intermolecular interactions that could cause the unusually high viscosity observed with mAb1. To confirm that these CDRs are the cause of high viscosity, the disclosed method was applied to investigate protein-protein interactions at high concentration of mAb2 which has the same amino acid sequence as mAb1 except for CDRs and has a low viscosity (FIGS. 4B, 4D, 4F, and 4H). Unlike mAb1, no differential deuterium uptake was observed between the high concentration of mAb2 samples and the low concentration mAb2 samples, further confirming that the CDRs of mAb1 caused the high viscosity at high concentrations.

TABLE 2
Relative deuterium uptake in non-CDR mAb1 peptide over time.

mAb1 non-CDR
Relative Deuterium Uptake (%)

| | | |
|---|---|---|
| Time point | 15 mg/ml | 120 mg/ml |

| | | | |
|---|---|---|---|
| 0 | hr | 0.0% | 0.0% |
| 4 | hrs | 36.7% | 33.2% |
| 24 | hrs | 41.7% | 38.6% |

TABLE 3
Relative deuterium uptake in LC-CDR mAb1 peptide over time.

mAb1 LC-CDR
Relative Deuterium Uptake (%)

| | | |
|---|---|---|
| Time point | 15 mg/ml | 120 mg/ml |

| | | | |
|---|---|---|---|
| 0 | hr | 0.0% | 0.0% |
| 4 | hrs | 49.8% | 39.1% |
| 24 | hrs | 65.6% | 52.2% |

### Example II: Differential Concentration-Dependent Viscosities Were Observed in mAb1 and mAb2

Two monoclonal antibody candidates, monoclonal antibody 1 (mAb1) and monoclonal antibody 2 (mAb2), each specific for the same therapeutic target and shares the same amino acid sequence except for the CDRs, were assessed for their potential development risks during the candidate selection stage. A significant difference in concentration-dependent viscosities was observed in mAb1 and mAb2 (FIGS. 1A and 1B). The viscosity of mAb1 increased dramatically with increasing protein concentration, while the viscosity of mAb2 increased only slightly with increasing protein concentration. In addition, mAb1 exhibited unusually high viscosity at concentrations above 100 mg/mL. No abnormal levels of higher molecular weight species were observed in both mAb1 and mAb1, indicating that the high viscosity was not caused by protein aggregation (data not shown). To elucidate the molecule mechanism causing of the high viscosity of mAb1 formulation, a dilution-free microdialysis plate-based HDX-MS method was developed to determine the amino acid residues involved in protein-protein interfaces which are likely responsible for the observed high viscosity (FIGS. 2A-2D). In this approach, HDX reactions took place using micro-dialysis cartridges to achieve dilution-free HDX. Compared to a previous dialysis-coupled method, this approach significantly reduces the sample amounts required and enables a higher throughput because of the 96-well microplate format. As a result, this method is suitable for candidate screening at an early stage of development when protein materials are limited.

While in the foregoing specification the invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

### Example III: CDR Regions of mAb1 Were the Protein-Protein Interfaces

The microdialysis plate-based HDX-MS was used to analyze deuterium uptakes in the high concentration (120 mg/mL) formulation versus the low concentration (15 mg/mL) formulation for both mAb1 and mAb2. 458 peptides were identified reproducibly from the HDX-MS analysis, resulting in a sequence coverage of 89.2% for the heavy chain (HC) and 100% for the light chain (LC) of mAb1 (data not shown). To compare the differential deuterium uptakes between the high concentration samples at 120 mg/mL and the low concentration samples at 15 mg/mL for mAb1 and mAb2, residual plots of identified mAb 1 and mAb2 peptides were created, in which the deuterium uptakes of the low concentration samples (15 mg/mL) were subtracted from the respective high concentration samples (120 mg/mL) (FIGS. 4C, 4D, 4G, and 4H). The residual plots show that most of the peptides have slightly lower deuterium uptakes at 120 mg/mL compared to deuterium uptakes at 15 mg/mL for both mAb1 and mAb2, likely due to the molecule crowding that makes proteins less accessible to D₂O at the high concentration and also reduces the diffusion rate that lowers the HD exchange rate at the high concentration. On average, we observed ˜5% systematically lower differential deuterium uptakes between the 120 mg/mL samples compared to 15 mg/mL samples. Due to these systemically differential deuterium uptakes, we considered a differential deuterium uptake of 10% (absolute value) or more as a significant differential deuterium uptake between the high concentration and the low concentration samples. For mAb1, we observed that the differential deuterium uptakes of HC CDR1 30-33, HC CDR2 50-54, HC CDR3 101-104, and LC CDR2 48-53 between the 120 mg/mL samples and the 15 mg/mL samples were significantly higher (≥10%, absolute value) compared to other peptide regions, indicating that these CDR regions were more protected under the high concentration compared to the low concentration. Thus, these CDR regions were likely at the interfaces of the mAb1 self-association. No significant differences in differential deuterium uptakes were observed at any sequence regions in mAb2, confirming that these CDR regions of mAb1 were at the interfaces of the mAb1 self-association.

### Example IV: Deuterium Uptake Results as a Function of HDX Labeling Time

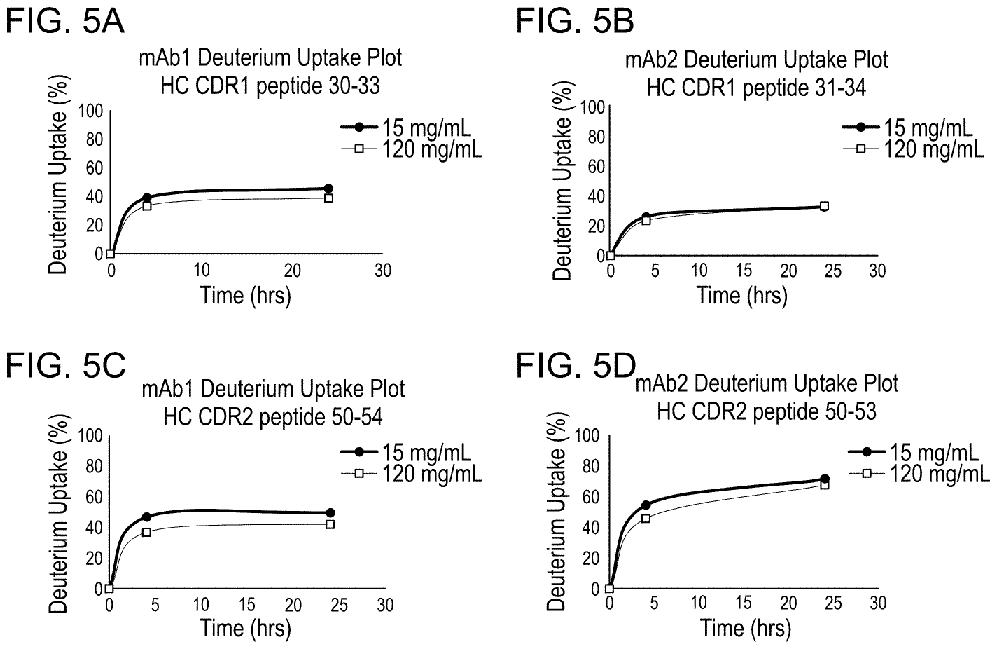

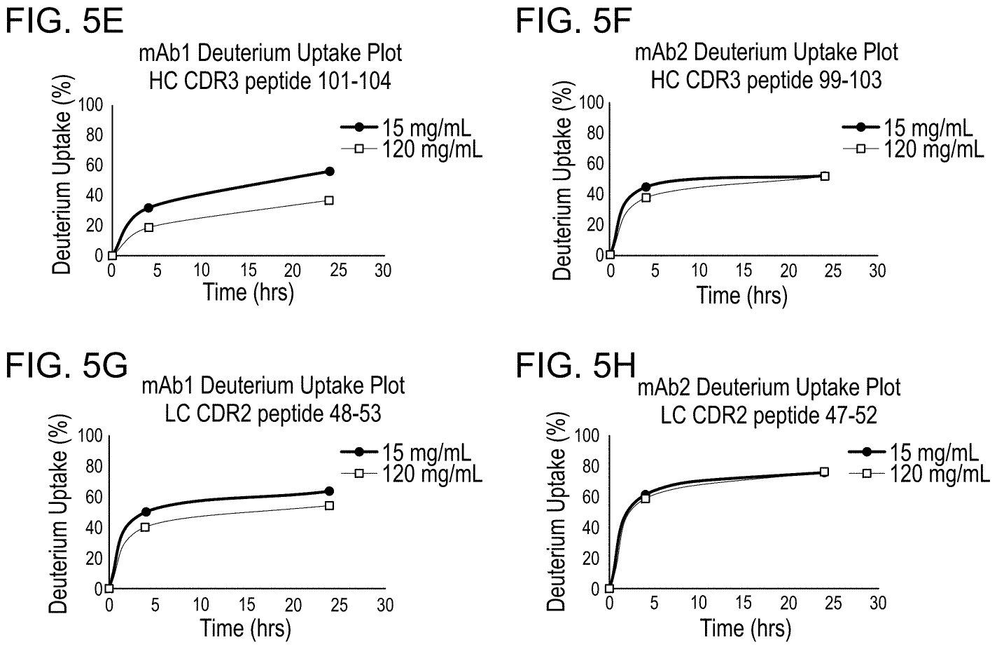

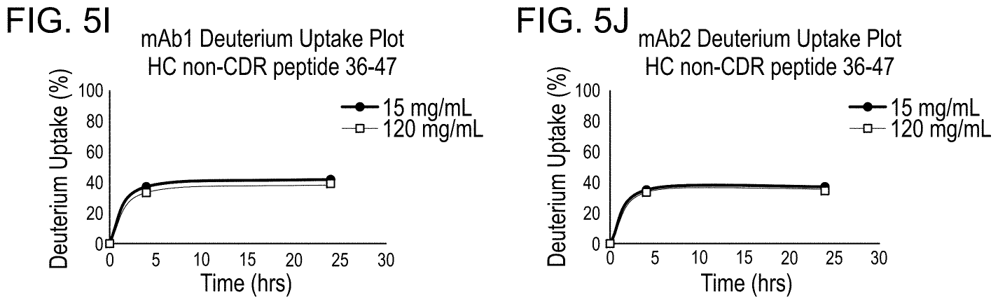

FIGS. 5A to 5J show the deuterium uptake results as a function of HDX labeling time for five representative peptides, including these four mAb1 CDR peptides (HC CDR1 30-33, HC CDR2 50-54, HC CDR3 101-104, and LC CDR2 48-53) and one mAb1 non-CDR peptide (HC non-CDR 36-47) as a comparison. The deuterium uptakes of five corresponding peptides at the same regions in mAb2 (HC CDR1 31-34, HC CDR2 50-53, HC CDR3 99-103, LC CDR2 47-52, and HC non-CDR 36-47) are also shown as a comparison. The deuterium uptakes of these peptides increased as the HDX reaction time increased until an equilibrium was reached at the 24-hour timepoint. FIG. 51 presents a representative mAb1 peptide that shows no significant difference in HDX kinetics between the high concentration and the low concentration samples, indicating this region was not involved in the interfaces of protein self-association. In contrast, FIGS. 5A, 5C, 5E, and 5G show that the four mAb1 CDR peptides (HC CDR1 30-33, HC CDR2 50-54, HC CDR3 101-104, and LC CDR2 48-53) had a significant differential deuterium uptake (≥10%, absolute value) between the high concentration and the low concentration samples, indicating that these regions were more buried in the high concentration samples compared to the low concentration samples and therefore were at the self-association interface. On the other hand, the corresponding regions in mAb2 showed very low differential deuterium uptakes (<5%, absolute value) (FIGS. 5B, 5D, 5F, 5H, 5J), indicating that no self-association was incurred in mAb2 high concentration samples.

### Example VI: Homology Model of mAb1 and mAb2

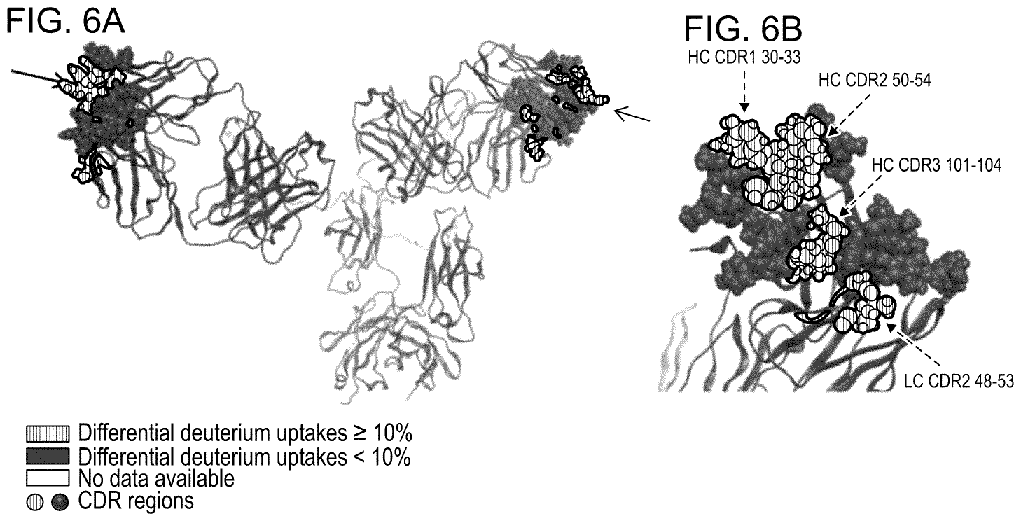

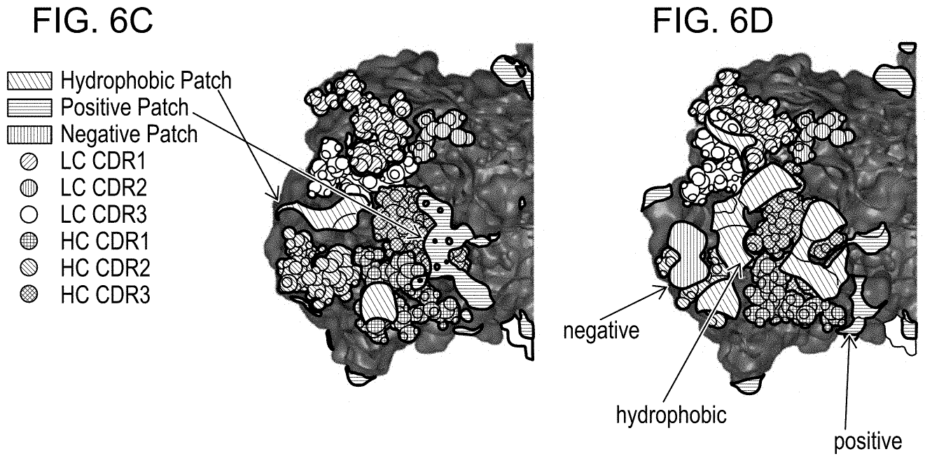

The HDX-MS results were mapped onto a homology model of mAb1 and mAb2 (FIGS. 6A to 6D). FIG. 6A shows the entire mAb1 and FIG. 6B shows a zoom-in view of the Fab region. The peptide regions in mAb1 associated with the self-association under the high concentration caused significant decreases in deuterium uptakes (≥10%, absolute value) are highlighted in red, while regions without significant differential deuterium uptake (<10%, absolute value) are colored in gray. The peptides that exhibited decreased deuterium uptakes at the high concentration compared to the low concentration were the solvent-exposed CDR regions and constituted the protein-protein interface for concentration-dependent reversible self-association of mAb 1. Analysis of protein surface patches of the Fab domains of mAb1 and mAb2 were shown in FIG. 6C and 6 D. The protein patch analyses reveal that the surface charge distributions and hydrophobicity of HC CDR1, CDR2, and CDR3 are different between mAb 1 and mAb2. mAb 1 HC CDR1 constructs a 50 Å² hydrophobic patch, HC CDR2 constructs a 70 Å² hydrophobic patch, and HC CDR3 and LC CDR2 construct a 140 Å² positively charged patch while mAb2 HC CDR1 and CDR3 construct a 170 Å² hydrophobic patch and HC CDR2 constructs an 80 Å² negatively charged patch. Therefore, the differences in the surface charge distributions and hydrophobicity of the CDR regions of mAb1 and mAb2 caused the reversible self-association of mAb1.

By analyzing deuterium uptake profiles of mAb1 and mAb2, it was observed that most of the peptides have slightly lower deuterium uptake (˜5%) in the 120 mg/mL samples compared to the deuterium uptakes in the 15 mg/mL samples for both mAb1 and mAb2 (FIGS. 4A-4H and FIGS. 5A-5J). It has been reported that the H/D exchange rate can vary with solution pH, temperature, solvent accessibility, and protein structure. In this study, solution pH and temperature were precisely controlled and were kept consistent across tested samples to ensure highly reproducible analyses. Thus, it is unlikely that solution pH and temperature were responsible for the observed H/D exchange rate difference between the 15 mg/mL and the 120 mg/mL samples. However, it is likely that the molecular crowding in the high concentration samples reduced the solvent accessibility and the flexibility of the protein backbone, leading to slower H/D exchange kinetics and slightly lower deuterium uptakes. In the high concentration samples, the ratio of D₂O to protein was lower than that in the low concentration samples. Also, the protein molecules were more crowded in the high concentration samples, reducing their accessibility to surrounding D2O. These two factors reduced the solvent accessibility and were likely the cause of the slightly lower deuterium uptakes observed in the mAb2 samples (FIG. 4C and 4D), where there was no concentration-dependent reversible self-association observed. Protein structure can also affect the H/D exchange rate, and the underlying mechanism was described by the Linderstrøm-Lang model⁴⁵. Based on the Linderstrøm-Lang model, the rate of H/D exchange depends on the intrinsic chemical exchange (kᵢₙₜ) and protein flexibility (k_{cl}/kₒₚ). Although we observed unusually high viscosity in mAb1 at 120 mg/mL, it is reported that viscosity difference has little influence on intrinsic chemical exchange. Backbone flexibility mainly depends on protein primary, secondary, tertiary, and quaternary protein structure. Reversible self-association, resulting from electrostatic interactions, van der Waals forces, or hydrophobic interactions, can affect the backbone flexibility of entire protein molecules. Indeed, the protein patch analyses revealed that the CDR regions of mAb1 and mAb2 exhibited differences in surface charge distributions and hydrophobicity (FIG. 6C). Thus, it is likely that the self-association reduced the backbone flexibility of mAb1 under the high concentration and resulted in slower H/D exchange kinetics and slightly lower deuterium uptakes (FIG. 4C and 4G).

The HDX-MS analysis revealed that certain peptides in mAb1 have increased protection against deuterium uptake in the high concentration samples (FIGS. 4A-4H). These peptides were therefore identified as the interaction interface for reversible self-association of mAb1. Specifically, four CDR regions, HC 30-33 (HC CDR1), HC 50-54 (HC CDR2), HC 101-104 (HC CDR3), and LC 48-53 (LC CDR2) in mAb1 showed a significant decrease in the deuterium uptake, demonstrating that these four CDR regions were involved in self-association that lead to the high viscosity at high concentration. In the similar CDR regions, deuterium uptake protection was not observed in the mAb2 samples, further confirming the involvement of mAb1 CDR residues in the mAb1 self-association. The involvement of CDR regions in the reversible self-association of antibodies was also reported in previous studies. For example, Bethea et al. used point mutations to demonstrate that F⁹⁹ and W¹⁰⁰ in the heavy chain CDR3 of a human IgG1 antibody were involved in protein self-association. In another study, Yadav et al. replaced charged residues in the CDR regions of a self-associating IgG1 antibody, leading to a dramatic decrease in solution viscosity²³. Similarly, Perchiacca et al. inserted two or more negatively charged residues at the edge of CDR3 of a single-domain (V_{H}) antibody, significantly reducing the protein aggregation caused by the clusters of hydrophobic residues within the CDR3. Likewise, Bethea et al. used the mutagenesis approach to identify that three residues ⁹⁹FHW¹⁰⁰ in the HC CDR3 of an IL13 mAb promoted self-association and aggregation²¹. Recently, Arora et al. reconstituted a lyophilized IgG1 mAb into 5 mg/mL and 60 mg/mL solutions using a D₂O labeling buffer followed by HDX-MS analysis and determined that HC CDR2 and LC CDR2 were at the protein-protein interface associated with concentration-dependent reversible self-association. These studies demonstrated that both charged residues and hydrophobic residues in the CDR regions could result in reversible self-association. Although mutagenesis analyses can accurately pinpoint the amino acid residues involved in self-association, point mutations can be time-consuming to conduct. The HDX-MS analyses could help locate the amino acid residues involved in self-association without conducting the time-consuming mutagenesis analyses.

Due to the increasing popularity of subcutaneous administration and demands for high concentration formulations, it is important to better understand the concentration-dependent reversible self-association of therapeutic mAb candidates. In this study, a dilution-free microdialysis HDX-MS was developed method to determine the amino acid residues at the self-association interfaces of mAb1. The method can help identify the amino acid residues at protein-protein interfaces before conducting the time-consuming mutagenesis analyses. Compared to the previously reported HDX-MS approaches, our microdialysis plate-based approach not only reduced the sample amount requirements, but also increased the analysis throughput. As a result, the microdialysis plate-based HDX-MS method, in combination with other orthogonal biophysical measurements, could be a suitable and powerful tool to use during the early stages of therapeutic mAb candidate selection and developability assessment to help understand reversible protein self-association and the causes of high viscosity.

The microdialysis plate-based HDX-MS method described herein can achieve HDX labeling without D₂O buffer dilution, allowing us to profile characteristic molecular interactions at different protein concentrations. The use of a microdialysis plate significantly reduced the consumption of samples and D₂O compared to traditional dialysis devices. The method was applied to an early stage developability assessment of two drug candidates, mAb1 and mAb2. While mAb 1 and mAb2 share the same amino acid sequence except for CDRs, mAb 1 had unusually high viscosity at high concentrations compared to mAb2. In mAb1, a significant decrease in deuterium uptake was observed between high concentration samples (120 mg/mL) and low concentration samples (15 mg/mL) at three heavy chain CDRs and light chain CDR2, while in mAb2, no differential deuterium uptake was observed between the high concentration samples and the low concentration samples. This result indicates that these CDRs in mAb1 were involved in intermolecular interactions, leading to unusually high viscosity in high concentration mAb1 samples.

The present invention may be embodied in other specific forms without departing from the spirit or essential attributes thereof and, accordingly, reference should be made to the appended claims, rather than to the foregoing specification, as indicating the scope of the invention.

1. A method for identifying regions in a protein that contribute to self-association of the protein, comprising:
microdialysing samples of the protein in a microdialysis cartridge against a buffer comprising deuterium for at least two different time periods;
subsequently quenching the microdialysis of the samples; and
analyzing the quenched samples in a hydrogen/deuterium exchange mass spectrometry system to determine surface charge distributions and hydrophobicity in regions of the protein in the sample that exhibit reduced levels of deuterium relative to other regions of the protein, wherein regions of the protein that exhibit reduced levels of deuterium contribute to self-association of the protein.

2. The method according to claim 1, wherein the protein is a monoclonal antibody.

3. The method according to claim 2, wherein the regions of the protein that exhibit reduced levels of deuterium are complementarity determining regions.

4. The method according to claim 1, where the microdialysing is performed at a concentration used in subcutaneous delivery.

5. The method according to claim 1, wherein surface charge distributions having positively charged patches contribute to self-association of the protein.

6. The method of claim 1, wherein samples of protein comprise between 10 mg/mL to 200 mg/mL of protein.

7. The method of claim 1, wherein samples of protein in the microdialysing step are in a buffer having a pH between 5.0 and 7.5.

8. The method of claim 1, wherein the samples of protein in the microdialysing step are in 10 mM Histidine at pH 6.0.

9. The method of claim 1, wherein the buffer comprising deuterium comprises 10 mM Histidine at pH 6.0.

10. The method of claim 1, wherein the microdialysis is performed at 2 to 6° C.

11. The method of claim 1, wherein at least one sample is microdialysed for 4 hours and at least another sample is microdialysed for 24 hours.

12. The method of claim 1, wherein the quenching step is performed at −2 to 2° C. for 1 to 5 minutes.

13. The method of claim 1, further comprising digesting the protein into peptides before mass spectrometry analysis.

14. The method of claim 1, wherein the protein is selected from the group consisting of an antibody, a fusion protein, a recombinant protein, or a combination thereof.

15. The method of claim 14, wherein the protein drug is a concentrated monoclonal antibody.

16. The method according to claim 2, wherein the monoclonal antibody is selected from the group consisting of abciximab, adalimumab, adalimumab-atto, ado-trastuzumab, alemtuzumab, alirocumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cemiplimab, cetuximab, denosumab, dinutuximab, dupilumab, durvalumab, eculizumab, elotuzumab, emicizumab-kxwh, emtansinealirocumab, evinacumab, evolocumab, fasinumab, golimumab, guselkumab, ibritumomab tiuxetan, idarucizumab, infliximab, infliximab-abda, infliximab-dyyb, ipilimumab, ixekizumab, mepolizumab, necitumumab, nesvacumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, sarilumab, secukinumab, siltuximab, tocilizumab, tocilizumab, trastuzumab, trevogrumab, ustekinumab, and vedolizumab.

17. The method according to claim 1, wherein protein is an Fc-fusion protein.

18. The protein drug produced by the method of claim 1.

19-33. (canceled)
